(19) United States
(12) Patent Application Publication
(10) Pub. No.: US 2014/0187595 A1
(43) Pub. Date: Jul. 3, 2014

(54) Methods and Compositions Comprising AMPK Activator (Metformin/Troglitazone) for the Treatment of Myotonic Dystrophy Type 1 (DM1)

(71) Applicant: INSERM (Institut National de la Sante et de la Recherche Medicale), Paris (FR)

(72) Inventors: Sandrine Baghdoyan, Evry Cedex (FR); Marc Peschanski, Evry Cedex (FR); Delphine Laustriat, Evry Cedex (FR); Jacqueline Gide, Evry Cedex (FR)

(73) Assignee: INSERM (Institut National de la Sante et de la Recherche Medicale), Paris (FR)

(21) Appl. No.: 14/152,585

(22) Filed: Jan. 10, 2014

Related U.S. Application Data
(63) Continuation of application No. 13/637,601, filed on Dec. 11, 2012.

(30) Foreign Application Priority Data
Apr. 2, 2010 (EP) 10305347.6

(51) Int. Cl. A61K 31/427; A61K 31/155

(52) U.S. Cl. CPC A61K 31/427; A61K 31/155
USPC 514/369; 514/635; 435/375

(57) ABSTRACT

The present invention relates to methods and compositions for the treatment of Myotonic Dystrophy type 1 (DM1) with an AMPK activator <eq.metformin or troglizazone>.

The present application is a continuation of U.S. patent application Ser. No. 13/637,601, which was filed on Dec. 11, 2012, which application was filed pursuant to 35 U.S.C. 371 as a U.S. National Phase application of International Patent Application No. PCT/EP2011/055099, which was filed Apr. 1, 2011, claiming the benefit of priority to European Patent Application No. 10305347.6, which was filed on Apr. 2, 2010. The entire text of the aforementioned applications is incorporated herein by reference in its entirety.

### FIELD OF THE INVENTION

The present invention relates to methods and compositions for the treatment of Myotonic Dystrophy type 1 (DM1).

### BACKGROUND OF THE INVENTION

Myotonic Dystrophy type 1 (DM1), the most common form of inherited muscular dystrophy in adults, is due to an unstable expansion of CTG triplet repeats in the 3′-untranslated region of the DMPK gene. This generates alternate splicing defects in a large number of genes ^{1,2}. The most explored molecular mechanism for those alterations is the abnormal function of the RNA-binding protein (RNA-BP) MBNL1, which is sequestered with the mutant RNA in intranuclear inclusions known as “foci”³. At least one other RNA-BP, CUGBP1, also shows functional alteration in DM1 cells, although not similar to MBNL1^{4,5,6}.

WO2009/105691 discloses a method for the treatment of myotonic comprising the administration of pentamidine to a subject in need thereof. Pentamidine reverses the splicing defects associated with myotonic dystrophy (see Warf et al. Proc Natl Acad Sci U.S.A. 2009;106(44):18551-6).

Mulders et al. Proc Natl Acad Sci U S A. 2009;106(33):13915-20 have shown that triplet-repeat oligonucleotide may reverse of RNA toxicity in myotonic dystrophy. However, to date, effective and specific ways of treating and/or preventing DM1 are scarce. Therefore, it is an object of the present invention to provide a method for treating and/or preventing DM1.

### SUMMARY OF THE INVENTION

The inventors have surprisingly demonstrated that AMPK activators restore splicing in myotonic dystrophy 1 cells via the RNA-binding protein ELAVL1. The present invention relates to an AMPK activator for use in a method for treating and/or preventing Myotonic Dystrophy type 1 (DM1).

The present invention also relates to a method for screening for compounds for treating and/or preventing DM1.

### DETAILED DESCRIPTION OF THE INVENTION

The present invention relates to an AMPK activator for use in a method for treating and/or preventing Myotonic Dystrophy type 1 (DM1) in a subject in need thereof.

The present invention also relates to the use of an AMPK activator for the manufacture of a medicament for treating and/or preventing Myotonic Dystrophy type 1 (DM1) in a subject in need thereof.

The present invention also relates to a method for treating and/or preventing Myotonic Dystrophy type 1 (DM1), comprising the step of administering an effective amount of an AMPK activator to a subject in need thereof.

By a “therapeutically effective amount” is meant a sufficient amount to be effective, at a reasonable benefit/risk ratio applicable to any medical treatment. It will be understood, however, that the total daily usage will be decided by the attending physician within the scope of sound medical judgment. The specific therapeutically effective dose level for any particular patient in need thereof will depend upon a variety of factors including the age, body weight, general health, severity of the pathology, symptoms extent, sex and diet of the patient, the time of administration, route of administration, the duration of the treatment; drugs used in combination or coincidental with the and like factors well known in the medical arts. For example, it is well known within the skill of the art to start doses of the compound at levels lower than those required to achieve the desired therapeutic effect and to gradually increase the dosage until the desired effect is achieved.

Adenosine 5′-monophosphate (AMP)-activated protein kinase (AMPK) activators are well known in the art (see for example fro review Zhang et al., Cell Metabolism 9, May 6, 2009).

Activation of AMPK may be induced by Indirect Activators such as Metformin, Thiazolidinediones such as troglitazone, rosiglitazone or pioglitazone, Adiponectin, Leptin, Ciliary Neurotrophic Factor (CNTF), Ghrelin/cCannabinoids, Interleukin-6, natural products such as alpha-Lipoic Acid alkaloids, bitter melon extracts, resveratrol, epigallocathechin gallate, berberine, quercetin, ginsenoside, curcumin, caffeic acid phenethyl ester, theaflavin . . .

Activation of AMPK may be induced by direct Activators such as A-769662 (Cool, B., et al. (2006). Cell Metab. 3, 403-416) or PT1 (Pang et al. (2008) J. Biol.Chem.283, 16051-16060).

Examples of patents disclosing AMPK activators are WO2009135580, WO2009124636, US20080221088, or EP1754483 which all disclose Thienopyridone derivatives, WO2008120797, EP2040702 which discloses imidazole derivatives, EP1907369 which discloses thiazole derivatives.

In an embodiment of the invention, the AMPK activator is metformin or a thiazolidinedione, such as for example troglitazone, rosiglitazone or pioglitazone.

Typically, two or more different AMPK activators may be used in combination for the treatment of DM1. By combining two or more different AMPK activators, the dosage of each AMPK activator may be reduced and thereby the risk of adverse reaction may be limited. This open an additional way of treatment for this kind of long term chronic administration as anticipated in formulation of marketed drugs including the association of metformin and one other member of the thiazolidinedione family.

Typically said two or more different AMPK activators may be administered simultaneously or sequentially. Said two or more different AMPK activators may be combined in a composition or as separate parts of a kit.

The present invention also relates to a composition for use as a medicament comprising two or more different AMPK activators.

The present invention also relates to a kit of parts comprising:

(A) a first AMPK activator; and

(B) a second AMPK activator, said first and second AMPK activator being different in term of chemical class.

Typically a first AMPK activator may be metformin and a second AMPK activator may be a thiazolidinedione, such as for example troglitazone, rosiglitazone or pioglitazone.

Metformin or thiazolidinedione have been used separately in some DM1 patients in order to treat insulin resistance, which is one of the multisystemic clinical features of DM1, together with myotonia, muscle weakness cataracts, cardiac conduction defects and multiple endocrinopathies (see Kouki et al, Diabet Med 2005;22(3):346-7; Kashiwagi,et al. Eur Neurol 1999; 41:171-172, Abe et al. Endocr. J. 2009; 56(7):911-3). Insulin sensitivity in skeletal muscle was shown to be decreased by 70% in patients with DM1 (Moxley et al., J Clin Invest, 1978) while whole body glucose disposal was reduced by 15-25% following insulin infusion (Moxley et al., J Clin Invest, 1984). Due to focal insulin resistance in muscle, the incidence of diabetes is only 5-9% in these patients (Matsumura et al., J Neurol Sci, 2009).

Insulin resistance is one of the multisystemic clinical features of DM1, its occurrence rate among DM1 patients is around 10% with late onset.

As used herein, the expression “focal insulin resistance” refers to insulin insensitivity of skeletal muscle with reduced glucose uptake.

As used herein, the expression “insulin resistance” or “systemic insulin resistance” refers to a physiological condition where the natural hormone, insulin, becomes less effective at lowering blood sugars. When fat and muscle cells fail to respond adequately to circulating insulin, blood glucose levels rise. Insulin resistance in muscle and fat cells reduces glucose uptake, whereas insulin resistance in liver cells results in reduced glycogen synthesis and storage and a failure to suppress glucose production and release into the blood. Insulin resistance normally refers to reduced glucose-lowering effects of insulin.

As used herein, the term “diabetes” refers to a metabolic disease in which a person has high blood sugar, either because the body does not produce enough insulin, or because cells do not respond to the insulin that is produced. This high blood sugar produces the classical symptoms of polyuria (frequent urination), polydipsia (increased thirst) and polyphagia (increased hunger).

As used herein the term “hyperglycemia” refers to a condition in which an excessive amount of glucose circulates in the blood plasma.

Without wishing to be bound by theory, it is thought that, in the method of the invention, the AMPK activator treats and/or prevents DM1 by restoring the splicing defects associated with the disease. Hence, the AMPK activator according to the invention treats and/or prevents the onset of the disease as a whole, rather than one or several symptoms of the disease.

In one embodiment of the invention, the subject is a presymptomatic DM1 patient. As used herein, the term “presymptomatic” refers to a patient whose DMPK gene contains an abnormal number of CTG repeats, but who does not yet present any clinical sign of the disease.

In one embodiment of the invention, said subject in need thereof is not suffering from insulin resistance.

In one embodiment of the invention, the subject does not suffer from diabetes.

The present invention also relates to a method for screening for compounds for treating and/or preventing DM1, comprising the following steps of:

a) adding the compound to be screened to a cell expressing ELAVL1; and

b) selecting the compound which enhances ELAVL1 nuclear import.

The person skilled in the art will be aware of standard techniques for implementing this method.

ELAVL1 (ELAV (embryonic lethal, abnormal vision, Drosophila)-like 1 (Hu antigen R)) is predominantly nuclear but shuttles between the nuclear and the cytoplasm.

Typically, in order to facilitate the localisation of ELAVL1 within the cell, fluorescence microscopy may be used. Typically DAPI (diamidino-2-phenylindole) may be used for the staining of the nucleus.

ELAVL1 may be directly labelled with a fluorescent protein such as GFP or YFP. Alternatively ELAVL1 may also be indirectly labelled with a fluorescent molecule by non covalent linkage, followed by immunohistochemistry. Typically ELAVL1 may be fused with a receptor or ligand and said fluorescent molecule may be fused with the corresponding ligand or receptor, so that the fluorescent molecule can non-covalently bind to ELAVL1. A suitable receptor/ligand couple may be the biotin/streptavidin paired member or may be selected among an antigen/antibody paired member. For example, ELAVL1 may be fused to a poly-histidine tail and the fluorescent molecule may be fused with an antibody directed against the poly-histidine tail.

Alternatively, cell fractionation followed by Western blot may be used.

Typically the ELAVL1 shuttling between the nuclear and the cytoplasm could be monitored by using a reporter construct containing a fusion of the ELAVL1 nucleocytoplasmic shuttling domain named HNS (Fan and Steitz, 1998, 15293-15298) and a fluorescent protein such as GFP.

### FIGURE LEGENDS

FIG. 1: ELAVL1 expression impacts on splicing impaired in DM1 at molecular and functional levels.

FIG. 1A, Analysis of INSR splicing (±exon 11) by quantitative PCR in DM1 MPCs 48 h after transfection with a set of 14 siRNAs targeting genes that encode proteins sharing at least one homologous RNA binding domain with either CUGBP1 or MBNL1 (means ±s.d., n=3). Arrows indicate the positive control MBNL1 siRNA that exacerbated the splicing defect, and the ELAVL1 siRNA that resulted in the opposite efficient reversal of the INSR splicing defect. Conversely, three siRNAs had MBNL1 siRNA-like effects, namely BRUNOL4, ELAVL4 and HNRNPF.

FIG. 1B. Similar ELAVL1 siRNA effects were obtained in WT and DM1 MPCs and human adult fibroblasts (means ±s.d., n=3).

FIG. 1C. 2-deoxy-D-glucose uptake plotted as absolute uptake (fmol/min/mg protein) in WT and DM1 MPCs 48 h after transfection with the indicated siRNAs (means ±s.d., n=2).

FIG. 1D. RT-PCR analysis of exogenous INSR (±exon 11) and

FIG. 1E cTNT splicing (±exon 5) in WT and DM1 MPCs cotransfected with the indicated expression vectors and the pSG (d) or pRG6 (e) minigenes respectively (means ±s.d., n=3).

FIG. 2: Blockade of nuclear import of ELAVL1 aggravates the ratio of insulin receptor isoforms.

FIG. 2A. Schema explaining ELAVL1 cytoplasmic fraction enrichment through the silencing of KPNA2 and TNPO2 transporters by RNA interference.

FIG. 2B. Quantitative PCR analysis of endogenous INSR splicing (±exon 11) 48 h after transfection of siRNAs targeting KPNA2 and TNPO2 in WT and DM1 MPCs (means ±s.d., n=3).

FIG. 3: Activators of AMPK that enhance ELAVL1 nuclear import restore INSR and cTNT DM1-impaired splicing.

FIG. 3A, A 24 h-treatment with increasing concentrations of metformin partially rescues INSR splicing (±exon 11) analyzed by quantitative PCR in DM1 MPCs (means ±s.d., n=3).

FIG. 3B, Metformin at 25 mM also restores exogenous cTNT splicing (±exon 5) as analyzed by RT-PCR in DM1 MPCs previously transfected with pRG6 minigene (means ±s.d., n=3).

FIG. 3C, A 72 h-treatment with 25 mM metformin results in ELAVL1 nuclear enrichment in WT and DM1 MPCs. Nuclear proteins (20 μg) from whole cell lysates were subjected to Western blot analysis to monitor the expression of ELAVL1 (left panel). Hybridization using antibody against Lamin A/C was carried out to control the quality of the fractionment procedure and the uniformity of nuclear samples loading. Three independent experiments were conducted and showed similar results. The nuclear ELAVL1 expression was estimated as a relative ratio of the intensity of ELAVL1 to Lamin A/C bands in each lane (right panel). Bands intensity was measured using Image J software.

FIGS. 3D and 3E, Quantitative analysis of INSR splicing in DM1 MPCs demonstrates that the association of metformin to troglitazone enabled to reach a maximal splicing rescue after 24 h of treatment (FIG. 3D) and that metformin corrective effect is maintained after 10 days of repeated treatment (means ±s.d., n=3) (FIG. 3E).

FIG. 4 Activators of AMPK restore splicing defects in vitro in cells obtained from DM1 patients and in vivo in mice

FIGS. 4A and 4B. Effects of AMPK activators on INSR splicing defect in immortalized myoblasts or freshly isolated peripheral blood lymphocytes (PBLs) from DM1 patients.

FIG. 4C. Metformin's ability to rescue missplicing in C57BL/6NCr1 mouse.

### EXAMPLE

We have made use, for the present study, of human pluripotent stem cell lines derived from embryos that displayed the mutant DMPK gene, as characterized during pre-implantation genetic diagnosis⁷. Cells of those DM1 lines differentiated along the mesodermal lineage⁸ exhibited foci and abnormal splicing of the insulin receptor (INSR) gene, allowing us to challenge 15 different RNA-binding proteins (RNA-BP) through a siRNA screen. Four of them impacted the ratio of INSR isoforms, out of which only one, ELAVL1, in a positive way toward normalization. This effect was confirmed in adult patients' samples, while ELAVL1 overexpression conversely exacerbated the splicing defect. Negative effect of ELAVL1 overexpression was mimicked by blockade of its nuclear shuttling through importins. Accordingly, AMPK activators —metformin and troglitazone⁹—that positively target importins demonstrated long-lasting corrective effects on INSR splicing. As a similar correction of abnormal splicing was also observed for cardiac troponin, targeting ELAVL1 through AMPK activators reveals clinically-relevant in DM1 patients beyond their classical use to treat glucose-related dysfunction.

Three stem cell lines were made available to us after derivation from embryos characterized as gene-carriers for the mutant DMPK gene, with original repeat numbers of about 250 (VUB19_DM1), 500 (VUB03_DM1)⁷ and 900 (VUB24_DM1) that secondarily extended over time. All three cell lines could be expanded at the undifferentiated stage and coaxed into the mesodermal lineage using a protocol⁸ that leads in two to three weeks to a phenotypically homogeneous population of cells that can self-renew without phenotypic changes for at least 15 passages and we call MPCs (for “mesodermal precursor cells”). These cells display many features commonly associated to bone marrow-derived adult mesenchymal stem cells⁸. At that stage, in situ hybridization using probes specific for the mutant DMPK RNA showed intranuclear aggregates that co-registered with focal accumulation of immunoreactive MBNL1, thus replicating the foci that are the main morphological features of DM1 cells. Parallel analysis of the insulin receptor isoforms using selective PCR revealing inclusion (INSR-B) or exclusion (INSR-A) of exon 11 demonstrated a significantly increased proportion of the latter as compared to the former, again in keeping with the defect observed in patients with DM1. As there was no apparent difference in the replication of DM1 features among the cell lines, the one which exhibited the intermediate number of repeats (VUB03_DM1) was used as a representative in subsequent steps.

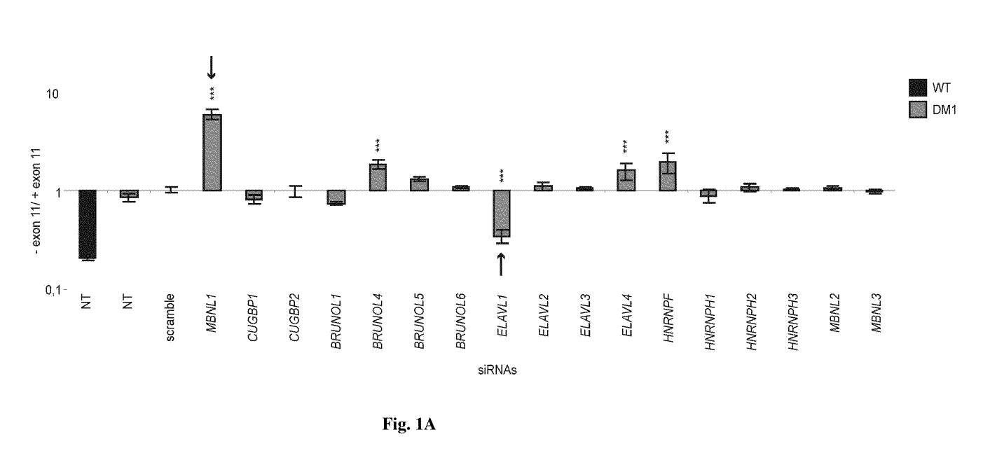

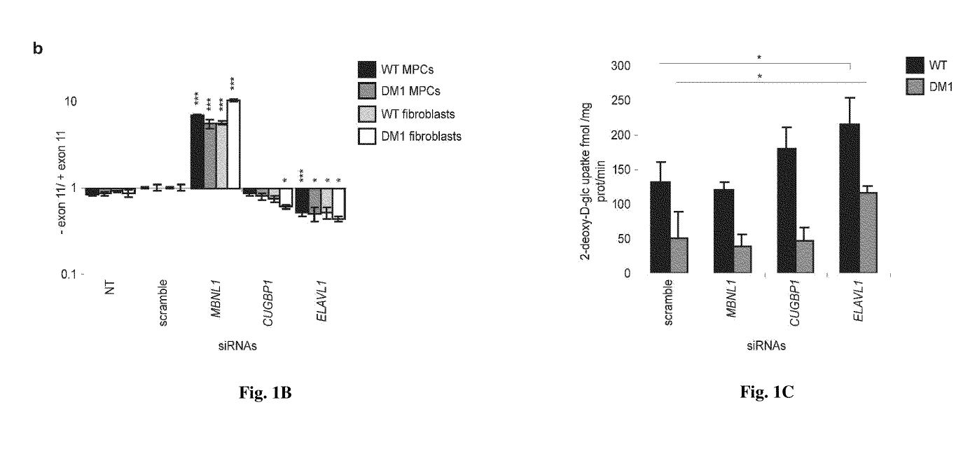

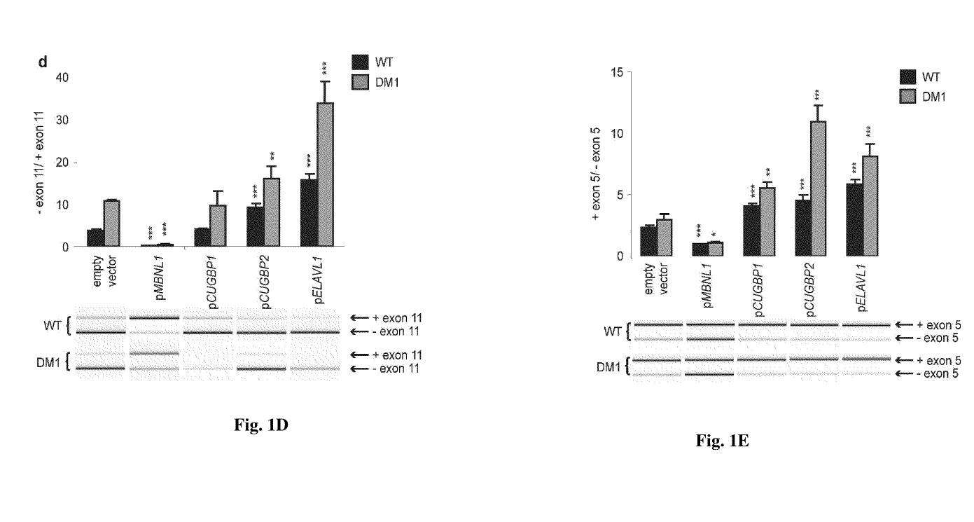

An RNA-interference screen was then performed, in the search for genes, the extinction of which would modify the INSR-A/INSR-B ratio in VUB03_DM1 cells. Candidate genes were first selected in silico on the basis of a sequence homology with at least one RNA binding domain of either CUBBP1, or MBNL1, i.e. RRM (RNA Recognition Motif) or C3H Zinc finger, respectively. Biological relevance was controlled by demonstrating their expression in MPCs and the assay technically validated by quantifying their extinction following application of the appropriate siRNA. Fourteen genes were thus selected in addition to MBNL1, CUGBP1 and CUGBP2 that were used as controls, and impact of their extinction on the INSR-A/INSR-B ratio measured using quantitative PCR (FIG. 1A). In keeping with the literature^{10,11}, a positive control was readily obtained using an siRNA targeting MBNL1 that strongly exacerbated the already abnormal ratio, whereas siRNA targeting CUGBP1 or CUGBP2 had no effect. Extinction of four of the fourteen assayed genes was associated with a statistically significant impact on the INSR-A/INSR-B ratio. Three of them, namely BRUNOL4, ELAVL4 and HNRNPF, partially exacerbated the defect, up to 30% of MBNL1 values. Conversely, only the knock-down of one, ELAVL1 (HuR) reversed it, but its effect was very strong, with a rescue of 80% of the pathological repartition between the two isoforms (p<0.001), in the absence of any effect on the overall INSR gene expression.

Because ELAVL1 thus appeared as the most active candidate, subsequent steps focused on it. The relevance of the results obtained in the embryonic stem cells-derived model to the actual DM1 pathology was first checked by confirming the corrective effect of ELAVL1 extinction in fibroblasts obtained from adult patients (FIG. 1B). In parallel, it was observed that these effects were not restricted to cells exhibiting an abnormal INSR-A/INSR-B ratio, as extinction of ELAVL1 also facilitated inclusion of exon 11 in wild-type cells, either of embryonic or adult origin. Extinction of MBNL1 had the opposite effect, as already described^{10,11} (FIG. 1B). As expected, overexpression of ELAVL1 using a co-transfection method with the pSG minigene, a reporter of INSR alternative splicing², had the opposite effect to extinction, resulting in an increased INSR-A/INSR-B ratio (p<0.001), again irrespective of the presence of the mutation (FIG. 1D,).

The corrective effects of ELAVL1 extinction was observed on other DM1-related abnormalities. First it was checked that another splicing defect could also be restored. TNNT2 (cTNT) was chosen because, opposite to INSR, abnormal splicing in DM1 leads to the inclusion of an exon (exon 5)⁴. As TNNT2 is not expressed in MPCs, cells were transfected with the RG6 minigene¹³ based upon the chicken orthologue. As for INSR, ELAVL1 was shown to regulate cTNT splicing in a way that opposed MBNL1, i.e. its knock-down decreased (data not shown) whereas its overexpression increased exon 5 inclusion (FIG. 1E). Second, DM1 patients exhibit insulinoresistance, as demonstrated in vitro by assaying glucose uptake. In keeping with original observations by others on patients' myotubes¹⁴, DM1 MPCs displayed a decreased insulin-stimulated glucose uptake at about 50% of their WT counterparts (FIG. 1C). Two days after treatment with ELAVL1 siRNA, glucose uptake increased in both WT and DM1 MPCs, up to normal level in the latter.

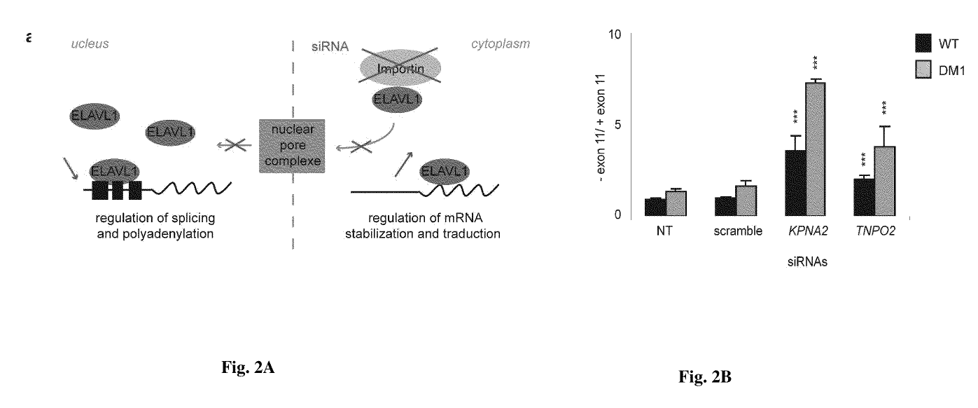

Altogether, these results pointed to ELAVL1 as a protein that exerted an action that strictly opposed that of MBNL1. The fact that this occurred as well in wild type as in DM1 cells pleaded against a mechanism that would act through intranuclear foci. This was fully confirmed by experiments that failed to demonstrate a co-localization of immunoreactive ELAVL1 with foci in DM1 cells, or else an effect of ELAVL1 extinction on either the number and size of DMPK aggregates themselves, or the co-localization of immunoreactive MBNL1 to foci. Once a direct action on foci was excluded, it remained to determine the impact of its subcellular localization. Like other RNA-BPs, ELAVL1 shuttles back and forth between nucleus and cytoplasm¹⁵. Its nuclear import is notably dependent on the action of the two importins encoded by the genes KPNA2 and TNPO2 and their extinction¹⁶, or expression as a deletion mutant¹⁷, significantly increases the concentration of ELAVL1 in the cytoplasm (FIG. 2A). Knock-down of these two genes using specific siRNAs significantly increased the INSR-A/INSR-B isoform ratio in both DM1 and WT MPCs (FIG. 2B), i.e. induced an effect comparable to the overexpression of ELAVL1, suggesting that the “anti-MBNL1” effect of ELAVL1 was linked to its relative cytoplasmic accumulation.

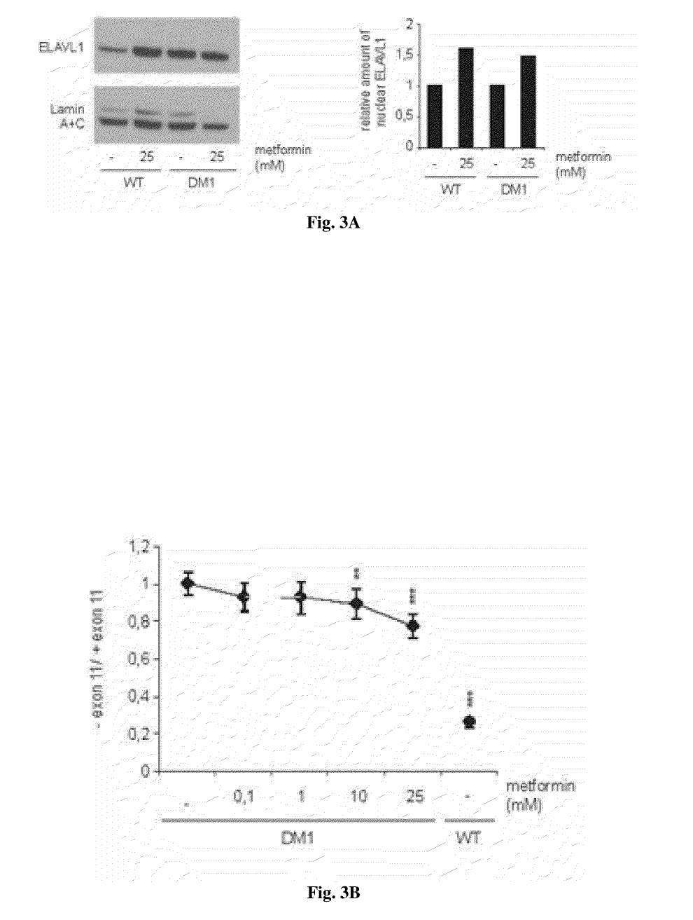

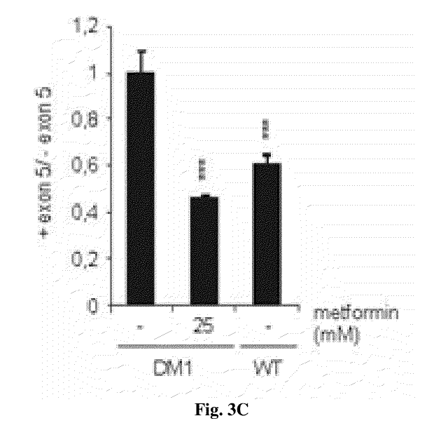

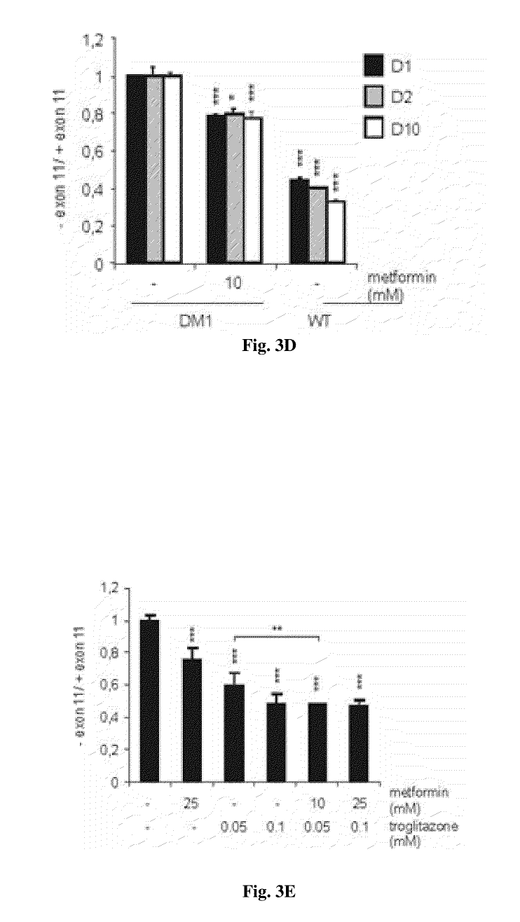

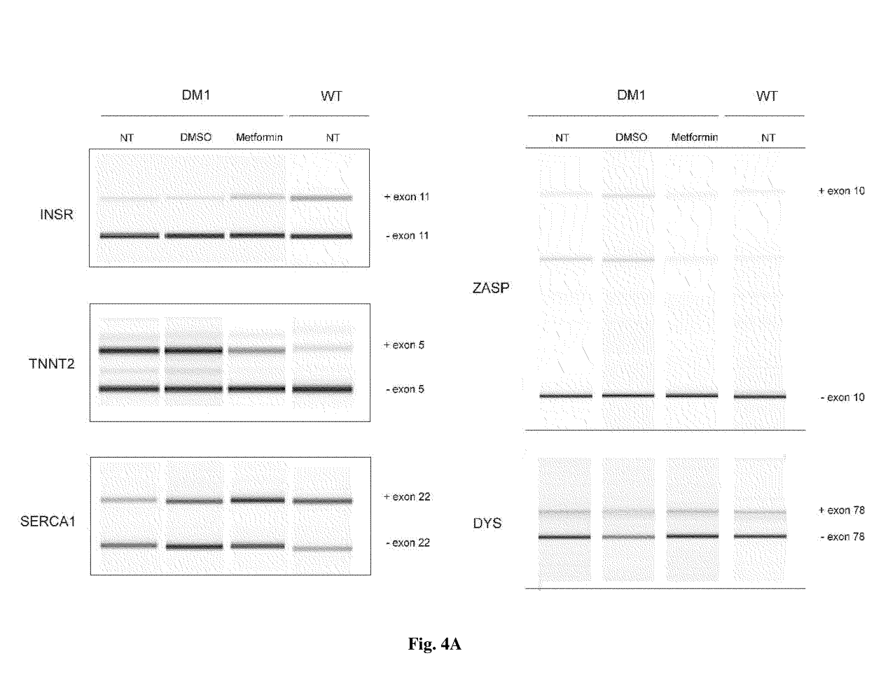

Conversely, this result suggested that the reverse effect may be obtained through increasing its relative nuclear accumulation. Such a result has been shown using AMPK activators that trigger ELAVL1 nuclear import through phosphorylation of Importin α1, the product of the KPNA2 gene¹⁸. This hypothesis was validated by the effect of metformin, a well known activator of AMPK. Indeed, metformin (25 mM) triggered a nuclear enrichment of ELAVL1 in both DM1 and WT MPCs (FIG. 3A). Treatment of DM1 MPCs at the same dose significantly decreased the INSR-A/INSR-B ratio by 30% (FIG. 3C). A corrective effect was also obtained with the pRG6 minigene, revealing restoration —i.e. decreased inclusion of exon 5 of cTNT splicing (FIG. 3C). Similar to ELAVL1 knock-down, metformin was also efficient in facilitating “MBNL1-related” splicing of the two genes in WT MPCs. Metformin is a widely prescribed anti-diabetic drug and its facilitation of nuclear import of ELAVL1 and parallel corrective effects on DM1-related abnormalities were encouraging in the search for a treatment for DM1. In DM1 cells, there was no observed toxicity when a dose of 10 mM inducing a maintained corrective effect on INSR splicing was repeated daily for up to 10 days (FIG. 3D). There has been one case-report in the literature¹⁹ of a patient with DM1 whose myotonia improved under treatment with another compound that activates AMPK, troglitazone, a member of the thiazolidinedione class of anti-diabetic drugs. In DM1 MPCs, troglitazone indeed revealed quite efficient at restoring INSR splicing since normal levels were reached with 100 μM (FIG. 3E). Metformin and troglitazone showed additive effects reaching normal levels for a combination of two submaximally effective doses of 10 mM and 50 μM, respectively.

The main result of this study is the identification of a protein, ELAVL1 (HuR) that counteracts MBNL1 effect on gene. This protein is druggable and its inhibition by siRNA as well as its facilitated nuclear import by AMPK activators has a corrective impact on splicing defects associated to myotonic dystrophy type 1. ELAVL1 does not act at the level of intranuclear foci, where MBNL1 is sequestered through binding to the mutant DMPK RNA, but rather corrective effects are linked to a decreased concentration of ELAVL1 in the cytoplasm.

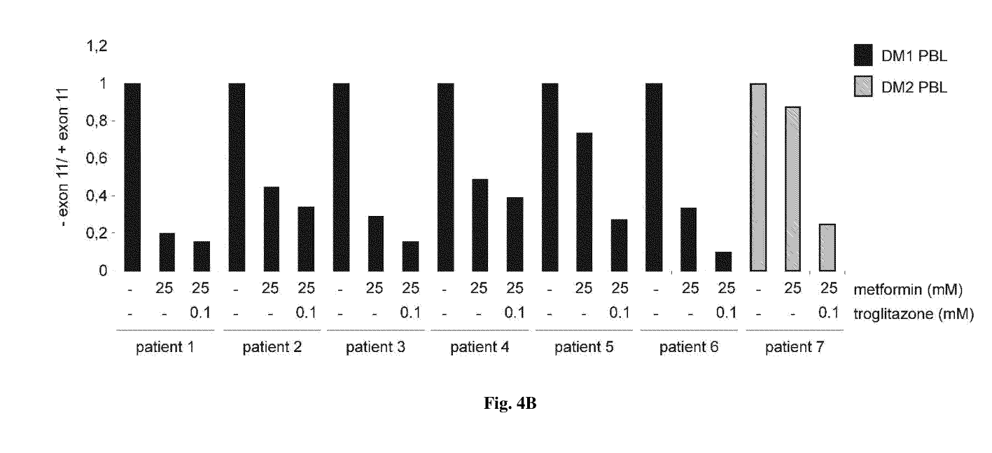

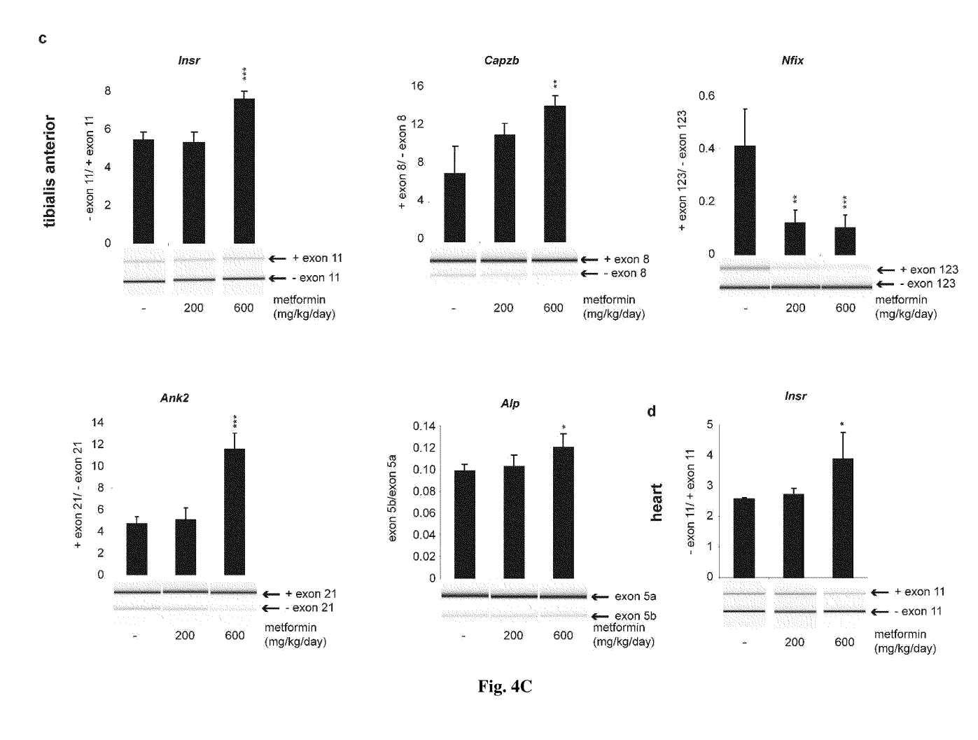

Clinical significance of these results was obtained by testing the effects of AMPK activators on immortalized myoblasts or freshly isolated peripheral blood lymphocytes (PBLs) from DM1 patients. Experiments performed in DM1 myoblasts confirmed the ability of Metformin to drive exon 11 inclusion on INSR transcript, exon 5 skipping on endogenous human cTNT transcript, exon 22 inclusion in SERCA1 transcript and exon 10 skipping from the ZASP transcript. In wild type and DM1 PBL samples, quantitative PCR analysis revealed a marked reduction of the INSR-A/INSR-B ratio in wild type and patients' PBLs after two days treatment with 25 mM metformin and 100 μM troglitazone (FIG. 4A-B). A similar effect was observed on PBLs obtained from one patient affected by Myotonic Dystrophy type 2 (DM2).

Metformin's ability to rescue missplicing was then tested in C57BL/6NCr1 mouse (FIG. 4C). Metformin was administered by gavage in 2 dosage regimens and missplicing of several pre-mRNA associated to DM1 that can be studied in wild type mouse were assayed. Enhancement of Ank2 exon 21 and Capzb exon 8 inclusion, INSR exon 11 and Nfix exon 123 exclusion or Alp exons 5a and 5b alternative splicing associated to a rescuing effect of DM1 missplicings in mouse skeletal muscle were observed. Metformin treatment at lower dose (200 mg/Kg/day) changed mainly Nfix splicing whereas a higher dose regimen (600 mg/Kg/day) can induce a significant splicing modification of the five pre-mRNA. No sign of toxicity was noted at the end of the treatment period for the two dosage regimens. Interestingly, the enhancement of INSR exon 11 exclusion was also observed in the heart at the higher dose demonstrating that Metformin's administration could have benefic effects not only on skeletal muscle but also on other organs affected by the DM1 through the rescue of missplicing.

### REFERENCES

Throughout this application, various references describe the state of the art to which the invention pertains. The disclosures of these references are hereby incorporated by reference into the present disclosure.
1 Du, H. et al., Nat Struct Mol Biol 17 (2), 187 (2010).
2 Ranum, L. P. and Cooper, T. A., Annu Rev Neurosci 29, 259 (2006).
3 Fardaei, M., Larkin, K., Brook, J. D., and Hamshere, M. G., Nucleic Acids Res 29 (13), 2766 (2001).
4 Philips, A. V., Timchenko, L. T., and Cooper, T. A., Science 280 (5364), 737 (1998).
5 Timchenko, L. T., Timchenko, N. A., Caskey, C. T., and Roberts, R., Hum Mol Genet 5 (1), 115 (1996).
6 Timchenko, N. A. et al., J Biol Chem 276 (11), 7820 (2001).
7 Mateizel, I. et al., Hum Reprod 21 (2), 503 (2006).
8 Mateizel, I. et al., Reprod Biomed Online 16 (5), 741 (2008).
9 Hardie, D. G., Annu Rev Pharmacol Toxicol 47, 185 (2007).
10 Dansithong, W., Paul, S., Comai, L., and Reddy, S., J Biol Chem 280 (7), 5773 (2005).
11 Paul, S. et al., EMBO J 25 (18), 4271 (2006).
12 Kosaki, A., Nelson, J., and Webster, N. J., J Biol Chem 273 (17), 10331 (1998).
13 Orengo, J. P., Bundman, D., and Cooper, T. A., Nucleic Acids Res 34 (22), e148 (2006).
14 Savkur, R. S., Philips, A. V., and Cooper, T. A., Nat Genet 29 (1), 40 (2001).
15 Hinman, M. N. and Lou, H., Cell Mol Life Sci 65 (20), 3168 (2008).
16 van der Giessen, K. and Gallouzi, I. E., Mol Biol Cell 18 (7), 2619 (2007).
17 Wang, W. et al., J Biol Chem 279 (46), 48376 (2004).
18 Wang, W. et al., Mol Cell Biol 22 (10), 3425 (2002).
19 Kashiwagi, K. et al., Eur Neurol 41 (3), 171 (1999).

1.-14. (canceled)

15. A method for treating and/or preventing Myotonic Dystrophy type 1 (DM1) in a presymptomatic DM1 patient, comprising administering an effective amount of an AMPK activator to the presymptomatic DM1 patient.

16. The method according to claim 15, wherein said patient is not suffering from insulin resistance.

17. The method according to claim 15, wherein said patient is not suffering from diabetes.

18. The method according to claim 15, wherein said AMPK activator is selected from the group consisting of Metformin, Thiazolidinediones, Adiponectin, Leptin, Ciliary Neurotrophic Factor (CNTF), Ghrelin/cCannabinoids, Interleukin-6, alpha-Lipoic Acid alkaloids, bitter melon extracts, resveratrol, epigallocathechin gallate, berberine, quercetin, ginsenoside, curcumin, caffeic acid phenethyl ester, theaflavin, A-769662, PT1, Thienopyridone derivatives, imidazole derivatives, and thiazole derivatives.

19. The method according to claim 15, wherein said AMPK activator is metformin or a thiazolidinedione.

20. The method according to claim 19, wherein said AMPK activator is metformin.

21. The method according to claim 19, wherein said thiazolidinedione is selected from the group consisting of troglitazone, rosiglitazone, and pioglitazone.

22. The method according to claim 15, comprising administering at least a first and a second AMPK activator to said patient, wherein the first AMPK activator is metformin and the second AMPK activator is a thiazolidinedione selected from the group consisting of troglitazone, rosiglitazone, and pioglitazone.

23. A method for correcting splicing defects in a myotonic dystrophy type 1 (DM1) cell, comprising contacting the DM1 cell with an effective amount of an AMPK activator.

24. The method according to claim 23, wherein said AMPK activator is selected from the group consisting of Metformin, Thiazolidinediones, Adiponectin, Leptin, Ciliary Neurotrophic Factor (CNTF), Ghrelin/cCannabinoids, Interleukin-6, alpha-Lipoic Acid alkaloids, bitter melon extracts, resveratrol, epigallocathechin gallate, berberine, quercetin, ginsenoside, curcumin, caffeic acid phenethyl ester, theaflavin, A-769662, PT1, Thienopyridone derivatives, imidazole derivatives, and thiazole derivatives.

25. The method according to claim 23, wherein said AMPK activator is metformin or a thiazolidinedione.

26. The method according to claim 25, wherein said AMPK activator is metformin.

27. The method according to claim 25, wherein said thiazolidinedione is selected from the group consisting of troglitazone, rosiglitazone or pioglitazone.

28. A method for modulating the ratio of the levels of expression of insulin receptor isoform, INSR-A to insulin receptor isoform, INSR-B in a cell, comprising contacting the cell with an effective amount of an AMPK activator.

29. The method of claim 28, wherein the amount of AMPK activator is effective to decrease the amount of ELAVL1 protein in the cytoplasm of the cell, relative to the amount of ELAVL1 protein in the cytoplasm of the cell in the absence of the AMPK1 activator.

30. The method of claim 28, wherein the amount of AMPK activator is effective to increase the import of ELAVL1 protein to the nucleus of the cell, relative to the import of ELAVL1 protein to the nucleus of the cell in the absence of the AMPK1 activator.

31. The method according to claim 28, wherein said AMPK activator is selected from the group consisting of Metformin, Thiazolidinediones, Adiponectin, Leptin, Ciliary Neurotrophic Factor (CNTF), Ghrelin/cCannabinoids, Interleukin-6, alpha-Lipoic Acid alkaloids, bitter melon extracts, resveratrol, epigallocathechin gallate, berberine, quercetin, ginsenoside, curcumin, caffeic acid phenethyl ester, theaflavin, A-769662, PT1, Thienopyridone derivatives, imidazole derivatives, and thiazole derivatives.

32. The method according to claim 28, wherein said AMPK activator is metformin or a thiazolidinedione.

33. The method according to claim 32, wherein said AMPK activator is metformin.

34. The method according to claim 32, wherein said thiazolidinedione is selected from the group consisting of troglitazone, rosiglitazone, and pioglitazone.
